(19) United States
(12) Patent Application Publication
(10) Pub. No.: US 2021/0353897 A1
(43) Pub. Date: Nov. 18, 2021

(54) DEVICES, SYSTEMS, AND METHODS FOR DISINFECTING AIR CONTAINING VIRUSES, BACTERIA, OR OTHER CONTAMINANTS

(71) Applicant: The Board of Regents of the Nevada System of Higher Education on Behalf of the University of Nevada,, Las Vegas, NV (US)

(72) Inventors: Zhiyong Wang, Henderson, NV (US); Jiulian Fu, Wuhan (CN)

(21) Appl. No.: 17/319,438

(22) Filed: May 13, 2021

Related U.S. Application Data
(60) Provisional application No. 63/024,202, filed on May 13, 2020.

(51) Int. Cl. A61M 16/10; B01D 46/00; B01D 46/42; A61L 9/20; A61L 9/18; A61L 9/16; A61L 9/015; A61M 11/00; A61M 1/16

(52) U.S. Cl. CPC A61M 16/1075; A61L 2209/132; B01D 46/4263; A61L 9/20; A61L 9/18; A61L 9/16; A61L 9/015; A61M 11/005; A61M 16/105; A61M 1/1698; B01D 2279/65; A61L 2209/16; A61L 2209/15; A61L 2209/111; A61L 2209/134; B01D 46/0028

(57) ABSTRACT

An apparatus used to disinfect viruses/bacteria contaminated exhaust air from devices, such as ventilators, CPAP, APAP, VPAP auto, BiPAO, ECMO, and also devices for air filtration used in cars, buildings, ships, planes, etc. is disclosed in this document. A heat source is used to burn the contaminated air at elevated temperatures, such as 100° C., 500° C., 1,000° C., and/or even more, in a confined environment to inactivate/destroy viruses/bacteria carried in the air. The heat sources can be, but not limited to, electrical, gases, infrared, microwave, and Ultraviolet (UV). After disinfection, the exhaust air from the apparatus is then released to the ambient environment, or to the next chamber for further treatment.

### CROSS-REFERENCE TO RELATED APPLICATION

This application claims priority to and the benefit of the filing date of U.S.

Provisional Patent Application No. 63/024,202, filed May 13, 2020, which is incorporated herein by reference in its entirety.

### FIELD

This application is directed to devices, systems, and methods for treating and eliminating air contaminants such as viruses and/or bacteria.

### BACKGROUND

Viruses have been responsible for many destructive outbreaks in human history. The flus of 1918, 1957, and 1968, SARS, MERS Ebola, and Covid-19 are all caused by viruses that encode their genetic material in ribonucleic acid (RNA). The zombielike existence of RNA viruses makes them easy to catch but hard to kill.

Outside a host, viruses are dormant and can last for a long time. Laboratory research has shown that although SARS-CoV-2 may degrade in a few hours or less outside a host, some particles can remain viable—potentially infectious—on cardboard for up to 24 hours and on plastic and stainless steel for up to nine days. In 2014, a virus frozen in permafrost for 30,000 years was able to infect an amoeba after being revived in a lab. Table 1 summarizes the number of hours different coronaviruses survive in air and on different surfaces.

TABLE 1
Summary of the number of hours coronaviruses
survive in air and on different surfaces

| | SARS-CoV-2 | SARS-CoV-1 | MERS-CoV-1 | HCoV |
|---|---|---|---|---|
| Air | 3 | 3 | — | — |
| Paper | — | 96 | — | — |
| Cardboard | 24 | 8 | — | — |
| Wood | — | 96 | — | — |
| Copper | 4 | 8 | — | — |
| Glass | — | 96 | — | 120 |
| Ceramic | — | — | — | 120 |
| Plastic | >72 | 216 | 48 | 144 |
| Steel | 48 | 48 | 48 | 120 |

SARS-CoV-2: causing COVID-19
SARS-CoV-1: caused SARS outbreak in 2003
MERS-CoV-1: caused MERS outbreak in 2012
—: no data available

Among RNA viruses, coronaviruses—named for the protein spikes that adorn them like points of a crown—are unique for their size and relative sophistication. They are three times bigger than the pathogens that cause dengue, West Nile, and Zika, and are capable of producing extra proteins that bolster their success. More particularly, coronaviruses contain a proofreading protein, which allows coronaviruses to fix some errors that happen during the replication process. They can still mutate faster than bacteria but are less likely to produce offspring riddled with detrimental mutations that they cannot survive.

Because Covid-19 is a relatively new species to humans, there are few scientific studies demonstrating the most effective disinfecting agents against Covid-19. However, the effectiveness of disinfectants against other coronaviruses is summarized below.

Soap and water are often the first line of defense to remove the virus from surfaces. Soap interferes with the fats in the virus shell and lifts the virus from surfaces, and the virus is then rinsed off by water.

The active ingredient in bleach—sodium hypochlorite—is very effective at killing viruses. The bleach works by destroying the protein and what is known as the RNA of the virus—this is the substance that gives the blueprint for making more virus particles when people become infected.

Surgical spirit is mostly made up of alcohol ethanol. Ethanol has been shown to kill coronaviruses in as little as 30 seconds. Like bleach, the alcohol destroys the protein and RNA that make up the virus.

Antiseptic works well on bacteria as well as on coronaviruses that infect mice and dogs—but it seems to make no difference to the spread of human coronavirus. Antiseptics work by disrupting the fats in pathogen cells, but SARS-CoV-2 does not contain fats. So far, there is no evidence that antiseptics can kill human Covid-19.

Hand sanitizers may kill viruses if the concentration of ethanol is over 70 percent. If the concentration of ethanol is too low, then hand sanitizers will not eliminate viruses effectively.

Hydrogen peroxide is approved by the FDA to decontaminate N95 face masks at hospitals during the Covid-19 pandemic. The system, called the STERRAD Sterilization System, uses vaporized hydrogen peroxide gas plasma sterilization, according to the agency.

Many other attempts, such as using ultraviolet germicidal irradiation and steam sterilization with moist heat, have been found to be effective for N95 face mask decontamination.

The above mentioned disinfectants offer little help to a patient using a ventilator or other life support devices, as each exhale of an infected person produces hundreds of tiny droplets which may contain thousands of viruses, which are released mostly to air. Once the viruses are airborne, they can survive for hours, which poses significant risk to nearby caregivers.

### SUMMARY

An apparatus used to disinfect contaminated exhaust air (e.g., air contaminated with viruses and/or bacteria) from one or more life support devices, such as ventilators, continuous positive airway pressure (CPAP) devices, automatic positive airway pressure (APAP) devices, variable positive airway pressure (VPAP) devices, bilevel positive airway pressure (BiPAP) devices, or extracorporeal membrane oxygenation (ECMO) devices, or from air circulation and/or filtration devices such as those used in cars, buildings, ships, planes, and the like, is disclosed herein. A heat source can be used to treat the contaminated air at elevated temperatures, such as from 100° C. to 500° C. or even more, in a confined environment to inactivate/destroy viruses/bacteria carried in the air. The heat sources can be, for example, but not limited to, electrical heat sources, gases, infrared heat sources, microwave heat sources, and/or Ultraviolet (UV) heat sources. After disinfection, the exhaust air from the apparatus can then be further treated or released to the ambient environment.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 is a perspective view of device in accordance with the embodiments disclosed herein.

FIG. 2 is an end view of the device as in FIG. 1.

FIG. 3 is a cross section of the device as in FIG. 1, taken in the plane labeled 3-3 in FIG. 2.

FIG. 4 is a side view of the device as in FIG. 1.

FIG. 5 is a cross section of the device as in FIG. 1, taken in the plane labeled 5-5 in FIG. 4.

FIG. 6 is a side view of a filter for use with the device as in FIG. 1.

FIG. 7 is a cross section of the filter as in FIG. 6, taken in the plane labeled 7-7 in FIG. 4.

FIG. 8 is a schematic diagram of a system comprising a device in accordance with embodiments disclosed herein.

### DETAILED DESCRIPTION

The present invention can be understood more readily by reference to the following detailed description and appendix, which include examples, drawings, and claims. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an,” and “the” include plural referents unless the context clearly dictates otherwise. Thus, for example, reference to “a heating element” can include two or more such heating elements unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

Studies have indicated that high temperature and high relative humidity significantly reduce the transmission of COVID-19. An increase of just one degree Celsius and a 1% relative humidity increase can substantially lower the transmission of the virus. Another study shows that the virus is stable for 3 weeks at room temperature in a liquid environment but easily killed by heat at 56° C. for 15 minutes. Similar studies have documented that H5N1 avian influenza virus is killed at 70° C. Among RNA viruses, including coronaviruses, proteins are part of the virus structure. Therefore, as further disclosed herein, the passage of exhaust air (e.g., from a ventilator) through a heating chamber at high temperatures can kill or lower viral loads.

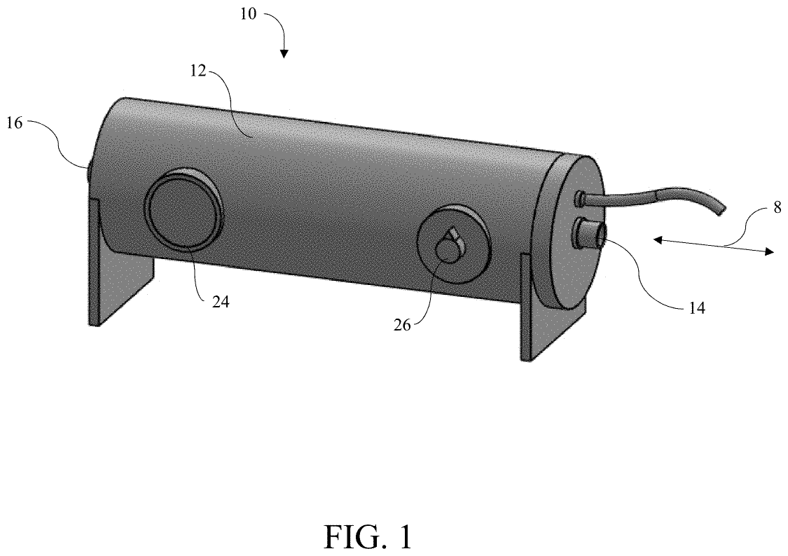

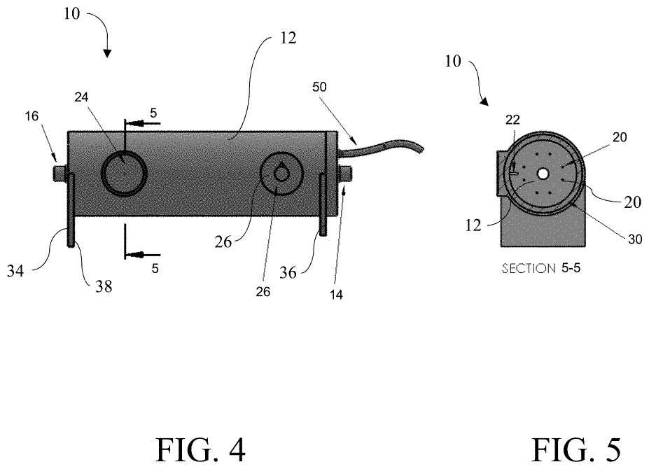

Disclosed herein, in various aspects and with reference to FIGS. 1-5, is a device 10 for disinfecting air. The device can comprise a chamber 12 having an inlet 14 and an outlet 16. The chamber 12 can further define a flow path 18 between the inlet 14 and the outlet 16. In some optional aspects, the inlet can be spaced from the outlet relative to a longitudinal axis 8. In various optional aspects, the chamber can be cylindrical. However, it is contemplated that other cross-sectional shapes of the chamber, such as rectangular cross-sections, can be used.

A heat source 20 can be configured to heat air or, at least, airborne viruses in air within the flow path 18. That is, it is contemplated that heating the viruses to or beyond a threshold temperature can kill the viruses. Optionally, to do so, the temperature of the air can be heated to the threshold temperature. In further aspects, it is contemplated that the temperature of the airborne viruses can be heated to at least a threshold temperature without the air itself being heated to the same threshold temperature using, for example, microwave radiation. It is contemplated that, in some optional aspects, heating the airborne viruses to at least the threshold temperature without heating the air itself can reduce energy costs.

The heat source 20 can be configured to heat airborne viruses in the air (and, optionally, the air) within the chamber to a threshold temperature or by a threshold amount. For example, in some aspects, the heat source can be configured to heat the airborne viruses (and, optionally, the air) within the chamber up to at least 50° C., at least 56° C., at least 75° C., at least 100° C., at least 150° C., at least 500° C., or at least 1000° C. between the inlet and outlet. Optionally, it is contemplated that the air can be permitted to exit the outlet 16 after reaching the threshold temperature. In further aspects, the heat source can be configured to heat air (and airborne viruses therein) within the chamber so that a temperature of the air within the chamber (or airborne viruses within the chamber) increases, between the inlet and the outlet, by a threshold amount, such as for example and without limitation, at least 50 degrees ° C., at least 75 degrees ° C., at least 100° C., at least 125° C., at least 150° C., at least 450° C., at least 500° C., at least 950° C., or at least 1000° C. In exemplary aspects, the heat source can be in communication with a timer (not shown), which can determine the duration of a period during which the heat source heats the air within the chamber. Optionally, in these aspects, the timer can be provided through a user interface and/or controller as further disclosed herein. Optionally, in further exemplary aspects, it is contemplated that the inlet and/or the outlet can comprise respective valves that can be selectively opened or closed to permit or restrict passage of air through the valve. For example, it is contemplated that a valve at the outlet can be configured to retain air within the chamber when the valve is in the closed position. When the air within the chamber reaches a threshold temperature or a threshold temperature increase, the valve at the outlet can be opened to permit passage of the air through the outlet (after destruction of the contaminants within the air can be confirmed or ensured based on the temperature or temperature increase).

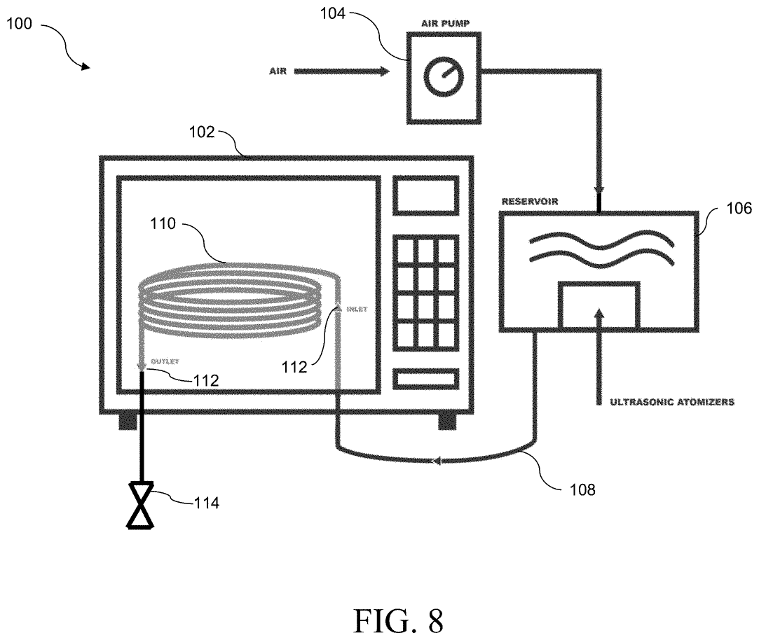

The inlet 14 of the chamber 12 of the device 10 can be in communication with an air source 104 (FIG. 8), such as, for example, a ventilator, a continuous positive airway pressure (CPAP) device, an automatic positive airway pressure (APAP) device, a variable positive airway pressure (VPAP) device, a bi-level positive airway pressure (BiPAP) device, an extracorporeal membrane oxygenation (ECMO) device, or a pump. In further aspects, the device 10 can be incorporated within an air handling system of a building or vehicle (e.g., car, plane, ship, etc.). For example, the device can be positioned in communication with the conventional air filtration, air circulation, and/or HVAC system of such a vehicle or building. In this way, the device 10 can disinfect the air to neutralize (e.g., inactivate or destroy) viruses and bacteria. Exhausted air from the outlet 16 can be released into the ambient environment or directed to other conduits or flow pathways for further processing and/or circulation.

In some optional aspects, the inlet 14 and outlet 16 of the chamber 12 can be on opposing longitudinal ends of the chamber. In some aspects, the flow path 18 can be straight or substantially straight. In further aspects, the flow path can be helical, winding, curved, zigzag, or undulating, or follow a switchback. For example, one or more baffles can at least partially define the flow path by directing the flow of air through the chamber 12. In some optional aspects, the flow path can have an operative length that is at least twice a longitudinal length of the device. In exemplary aspects, the inlet 14 can comprise a port that is complementary to an air tube or other conduit in fluid communication with the air source.

In some aspects, the heat source 20 can comprise at least one resistive heating element, such as, for example one or more wires, coils, plates, sheets, tape sections, or combinations thereof. In some aspects, the heat source 20 can receive power from a power source, such as, for example, a power cable 50 or a battery. In some aspects, the heating elements can extend along the longitudinal axis 8. Optionally, the at least one resistive heating element can comprise a plurality of longitudinally oriented heating elements. For example, in some aspects, the plurality of longitudinally oriented heating elements can be spaced about a circumference of the chamber. In other optional aspects, a plurality of heating elements can be spaced along the longitudinal axis 8. Optionally, in these aspects, at least one heating element can be positioned in an upper portion of the chamber, and at least one heating element can be positioned in a lower portion of the chamber. The required power output in watts of the heating elements can be estimated as P=SCFM*ΔT/3, where SCFM is the molar flow rate of gas in standard cubic feet per minute and ΔT is the desired temperature differential in Celsius between the input and the output. In further aspects, the heat source can comprise infrared radiation, microwave radiation, ultraviolet radiation, a gas, or other suitable heat source. In further aspects, the heat source can comprise a combination of such heat sources.

The device 10 can optionally comprise at least one thermocouple 22 or other temperature sensor. Optionally, one or more of the at least one thermocouple can be positioned proximate to the outlet 16. In some aspects, the device can comprise a display 24 (e.g., a dial display or LCD digital display) that is configured to display temperature measurements from the thermocouple 22. In some aspects, the device can comprise a user input device 26 (e.g., a dial, touchscreen or other user interface). In some optional aspects, a user can use the input device to control the amount of heat that the heat source provides, for example, by increasing the power to the heating elements. Optionally, it is contemplated that the input device can receive an input from a user indicative of a selected temperature within the chamber. In further aspects, the device 10 can comprise a controller (not shown) that is in communication with the thermocouple 22 (through wired or wireless communication) and configured to regulate the heat source 20 (via wired or wireless communication) to maintain a select air temperature at the thermocouple 22. In some aspects, the input device 26 can be in communication with the controller, and the inputs provided to the input device can be used to set a heating time and/or the select air temperature that the controller maintains. Optionally, the display 24 and/or the user input device can be secured or coupled to an outer surface of the device (positioned radially outward of the chamber). Optionally, in some aspects, it is contemplated that the controller can be in communication with actuators that are configured to open or close valves at the inlet or outlet of the chamber as disclosed herein. In these aspects, the controller can be configured to control opening or closing of the valves based on a timer and/or the temperature within the chamber.

The device 10 can comprise insulation 30 that surrounds the chamber. The device can optionally comprise a stand 34. The stand 34 can optionally support the device so that its outlet 16 is elevated above the inlet 14. For example, for an embodiment in which the inlet and outlet are spaced relative to the longitudinal axis 8, the longitudinal axis 8 can be maintained at an acute angle with respect to a horizontal surface. This can be achieved, for example with the stand having a front leg 36 (proximate the inlet 14) with a length that is shorter than a length of a rear leg 38 (proximate the outlet 16). Such a configuration can facilitate airflow through the chamber due to the rising of the heated air.

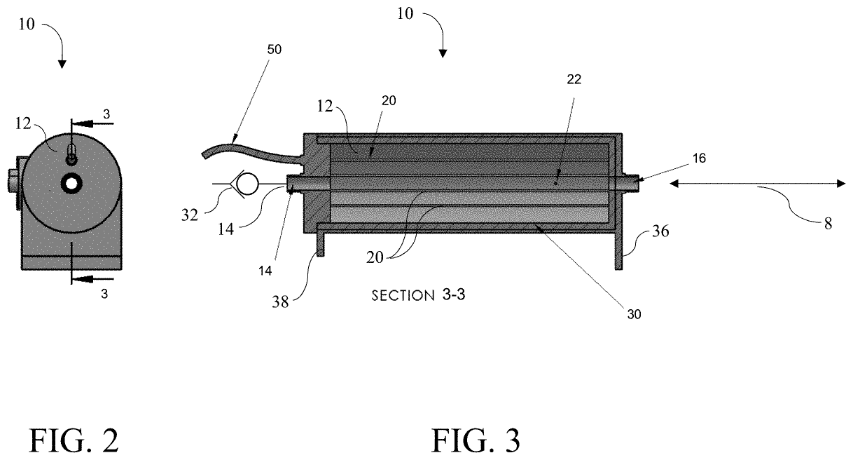

In some aspects, a one-way valve 32 (e.g., a check valve, shown as a schematic element in FIG. 3) can be provided to inhibit backflow through the inlet. In these aspects, it is contemplated that the one-way valve 32 can prevent backflow of contaminated air particles.

Air treated in the device 10 can be tested to ensure that the bacteria and viruses are sufficiently neutralized, for example, by quantitative real-time polymerase chain reaction, a commonly used test method by medical professionals. Viruses can also be detected by a biosensor. For example, in some aspects, the biosensor can comprise small structures of gold (e.g., nanoislands) on a glass substrate and DNA receptors that match specific RNA sequences of a virus (e.g., SARS-CoV-2) can be grafted onto the gold structures (nanoislands). The receptors on the sensor can be complementary sequences to the coronavirus's unique RNA sequences to help reliably identify the virus. Localized surface plasmon resonance can be used to detect the virus. The technology can use an optical phenomenon that happens in metallic nanostructures. Modulated incident light in specific wavelength ranges can create a plasmonic near-field around the nanostructure. Once molecules bind to the surface, the local refractive index in the plasmonic near-field changes. The sensor's plasmonic photothermal effect can produce localized heat when the nanostructure on the sensor is excited with a laser of a certain wavelength.

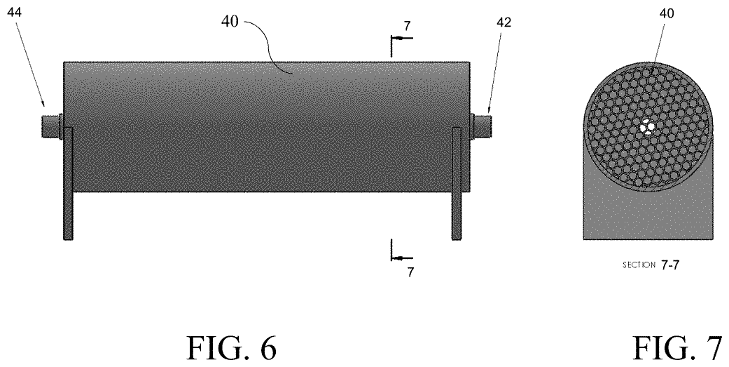

Referring to FIGS. 6 and 7, in some further aspects, a filter 40 can be positioned in communication with the outlet. The filter can have an inlet 42 and an outlet 44. Optionally, each of the inlet 42 and the outlet 44 can have a respective valve The filter 40 can optionally comprise nano-sized pores for filtering the outlet air. For example, it is contemplated that the filter 40 can prevent passage of particles having a size of greater than 100 nm. Optionally, the filter 40 can be coupled to the outlet of the chamber. In further aspects, the filter 40 can be provided as part of a separate treatment apparatus. Optionally, the separate treatment apparatus can be positioned in fluid communication with the outlet of the chamber. Alternatively, it is contemplated that the separate treatment apparatus can be positioned in fluid communication with the inlet of the chamber such that filtered air is received within the chamber. Additionally, or alternatively, when a further treatment apparatus is provided at the outlet or inlet side of the system, it is contemplated that the further treatment apparatus can provide ultraviolet germicidal irradiation or steam sterilization with moist heat. In further optional aspects, a cooling device, such as, for example, a heat exchanger having a coolant pumped therethrough or a heat sink, can be used to cool the air exiting the chamber or the further treatment apparatus before the air is released to the environment or directed to further downstream purposes, such as recirculation.

FIG. 8 illustrates a system 100 comprising a microwave device 102 that can be used for airborne virus disinfection. In various aspects, the microwave device 102 can be a conventional microwave oven. In further aspects, the microwave device can be a device configured for the purpose of disinfecting air. In some aspects, air can be delivered into an ultrasonic atomizer 106, optionally via a pump of an air source 104. Optionally, the ultrasonic atomizer 106 can define a vessel (e.g., a reservoir) that allows the air to mix with moisture produced by the ultrasonic atomizer 106. Air from the ultrasonic atomizer 106 can be provided to the microwave device 102 via a conduit 108 (e.g., a length of tubing). The conduit 108 can have a portion 110 within the microwave device. In various aspects, the portion 110 of the conduit 108 can have a flow path having a predetermined length within the microwave device 102. The portion 108 of the conduit 108 can optionally have a helical (e.g., looped), winding, curved, zig-zag, or undulating flow path, or follow a switchback flow path. In this way, the air travel distance inside the microwave device 102 can be controlled by the flow path (e.g., by selecting a diameter and a number of loops of tubing) inside the microwave device 102. Optionally, a metal mesh 112 can be provided at one or both of the tube inlet and outlet. The mesh 112 can permit air to pass through while inhibiting microwave energy from leaking out. The mesh 112 can be continuously conductive. The mesh 112 can comprise ferrous metal. The mesh can define openings therethrough that are sized to reflect microwave radiation (e.g., about 1/10 to ¼ of the wavelength of the microwave radiation). The microwave device can heat the air and airborne viruses to a high temperature, such as, for example, at least 150° C. As can be understood, microwave radiation can be provided at various powers that can, in some optional aspects, heat the air and/or airborne viruses at a rapid rate. Moisture produced by the ultrasonic atomizer can accelerate the heating process of viruses in the air. After the air is disinfected by the microwave irradiation, the air can be released (e.g., to ambient air) via an outlet. The outlet can optionally be controlled by valve 114. The valve 114 can optionally have at least an open and a closed configuration.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A device comprising: a chamber having an inlet and an outlet, wherein the chamber defines a flow path between the inlet and the outlet; and a heat source that is configured to heat air within the flow path so that a temperature of air within the chamber reaches at least 100° C.

Aspect 2: The device of aspect 1, wherein the heat source is configured to heat the air within the flow path so that a temperature differential between the air entering the inlet and the air exiting the outlet is at least 150° C.

Aspect 3: The device of aspect 2, wherein the heat source is configured to heat the air within the flow path so that a temperature differential between the air entering the inlet and the air exiting the outlet is at least 500° C.

Aspect 4: The device of aspect 3, wherein the heat source is configured to heat the air within the flow path so that a temperature differential between the air entering the inlet and the air exiting the outlet is at least 1000° C.

Aspect 5: The device of any one of the preceding aspects, further comprising at least one thermocouple that is in communication with the flow path.

Aspect 6: The device of any one of the preceding aspects, wherein the heat source comprises at least one of an electric heating element, infrared radiation, microwave radiation, ultraviolet radiation, or a gas.

Aspect 7: The device of any one of the preceding aspects, wherein the flow path is one of straight, helical, switchback, winding, or undulating.

Aspect 8: The device of aspect 7, wherein the device has a longitudinal length, wherein the flow path has a length that is at least twice the longitudinal length of the device.

Aspect 9: The device of any one of the preceding aspects, wherein the inlet and outlet are spaced relative to a longitudinal axis, and wherein the device comprises at least one heating element extending along the longitudinal axis.

Aspect 10: The device of any one of the preceding aspects, further comprising a stand configured to support the chamber in a desired orientation.

Aspect 11: The device of aspect 10, wherein the inlet and outlet are spaced relative to a longitudinal axis, wherein the stand supports the device so that the longitudinal axis is at an acute angle with respect to a horizontal surface.

Aspect 12: The device of any one of the preceding aspects, further comprising insulation surrounding the chamber.

Aspect 13: The device of any one of the preceding aspects, further comprising a one-way valve that inhibits backflow through the inlet.

Aspect 14: A system comprising: a device having: a chamber having an inlet and an outlet, wherein the chamber defines a flow path between the inlet and the outlet; and a heat source that is configured to heat air within the flow path so that a temperature of air within the chamber reaches at least 100° C.; and an air source in communication with the inlet of the device.

Aspect 15: The system of aspect 14, wherein the air source is one of a ventilator, a CPAP device, an APAP device, a VPAP device, a BiPAP device, an ECMO device, or air from a vehicle or building.

Aspect 16: The system of aspect 14 or aspect 15, further comprising a filter in fluid communication with the device.

Aspect 17: The system of any one of aspects 14-16, further comprising an ultrasonic atomizer that is configured to provide moisture to the air delivered to the flow path.

Aspect 18: A method comprising: receiving contaminated air at an inlet of a chamber of a device, the chamber defining a flow path between the inlet and an outlet of the chamber; heating, by a heat source that is configured to heat air within the flow path, the contaminated air to at least 100° C.; and exhausting the heated air at the outlet of the chamber.

Aspect 19: The method of claim 18, wherein heating the contaminated air to at least 100° C. comprises heating the contaminated air to at least 500° C.

Aspect 20: The method of claim 18, wherein receiving the contaminated air comprises receiving the contaminated air from one of a ventilator, CPAP, APAP, VPAP auto, BiPAP, ECMO, or a vehicle or building.

Aspect 21: The method of claim 18, wherein the contaminated air comprises virus particles.

Although several embodiments of the invention have been disclosed in the foregoing specification and the following appendices, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed herein, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A device comprising:
a chamber having an inlet and an outlet, wherein the chamber defines a flow path between the inlet and the outlet; and
a heat source that is configured to heat air within the flow path so that a temperature of air within the chamber reaches at least 100° C.

2. The device of claim 1, further comprising at least one thermocouple that is in communication with the flow path.

3. The device of claim 1, wherein the heat source comprises at least one of an electric heating element, infrared radiation, microwave radiation, ultraviolet radiation, or a gas.

4. The device of claim 1, wherein the flow path is one of straight, helical, switchback, winding, or undulating.

5. The device of claim 4, wherein the device has a longitudinal length, wherein the flow path has a length that is at least twice the longitudinal length of the device.

6. The device of claim 1, wherein the inlet and outlet are spaced along a longitudinal axis, and wherein the device comprises at least one heating element extending along the longitudinal axis.

7. The device of claim 1, further comprising a stand configured to support the chamber in a desired orientation.

8. The device of claim 7, wherein the inlet and outlet are spaced along a longitudinal axis, wherein the stand supports the device so that the longitudinal axis is at an acute angle with respect to a horizontal surface.

9. The device of claim 1, further comprising insulation surrounding the chamber.

10. The device of claim 1, further comprising a one-way valve that inhibits backflow through the inlet.

11. A system comprising:
a device having:
a chamber having an inlet and an outlet, wherein the chamber defines a flow path between the inlet and the outlet; and
a heat source that is configured to heat air within the flow path so that a temperature of air within the chamber reaches at least 100° C.; and
an air source in communication with the inlet of the device.

12. The system of claim 11, wherein the air source is one of a ventilator, a CPAP device, an APAP device, a VPAP device, a BiPAP device, an ECMO device, or air from a vehicle or building.

13. The system of claim 12, further comprising a filter in fluid communication with the device.

14. The system of claim 12, further comprising an ultrasonic atomizer that is configured to provide moisture to the air delivered to the flow path.

15. A method comprising:
receiving contaminated air at an inlet of a chamber of a device, the chamber defining a flow path between the inlet and an outlet of the chamber;
heating, by a heat source that is configured to heat air within the flow path, the contaminated air to at least 100° C.; and
exhausting the heated air at the outlet of the chamber.

16. The method of claim 15, wherein receiving the contaminated air comprises receiving the contaminated air from one of a ventilator, CPAP, APAP, VPAP auto, BiPAP, ECMO, or a vehicle or building.

17. The method of claim 15, wherein the contaminated air comprises virus particles.

18. The method of claim 17, wherein heating the contaminated air to at least 100° C. comprises heating the viruses particles to at least 150° C.

19. The method of claim 15, wherein the heat source is configured to heat the air within the flow path so that a temperature differential between the air entering the inlet and the air exiting the outlet is at least 150° C.

20. The method of claim 15, wherein an air source is in communication with the inlet of the device, the air source being one of a ventilator, a CPAP device, an APAP device, a VPAP device, a BiPAP device, an ECMO device, or air from a vehicle or building, wherein the method further comprises providing, by an ultrasonic atomizer, moisture to air delivered to the flow path by the air source.
